(19) United States
(12) Patent Application Publication
(10) Pub. No.: US 2012/0004636 A1
(43) Pub. Date: Jan. 5, 2012

(54) HEMOSTATIC FIBROUS MATERIAL

(76) Inventor: Denny Lo, Bethlehem, CT (US)

(21) Appl. No.: 13/175,380

(22) Filed: Jul. 1, 2011

Related U.S. Application Data
(60) Provisional application No. 61/361,284, filed on Jul. 2, 2010.

(51) Int. Cl. A61M 37/00; A61P 7/04; A61K 33/06; A61B 19/02; A61K 9/70

(52) U.S. Cl. 604/500; 206/440; 424/402; 424/684

(57) ABSTRACT

This disclosure relates to devices for promoting the clotting of blood in human beings or animals, or hemostatic devices. These devices may comprise a fibrous material or materials comprising one or more fibers such as a gauze or a cloth. Some of the fibers in these materials or devices may comprise a macromolecular material and a hemostatic hemostatic additive material such as kaolin or another clay.

### CROSS-REFERENCE TO RELATED APPLICATIONS

This application claims the benefit of U.S. Provisional Application No. 61/361,284, filed Jul. 2, 2010, which is incorporated by reference herein in its entirety.

### BACKGROUND OF THE INVENTION

1. Field

Various embodiments disclosed herein relate generally to hemostatic methods, devices and materials, and specifically to hemostatic fibrous materials.

2. Description of the Related Art

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, wounds can be inflicted as the result of trauma. Often bleeding is associated with such wounds. In some circumstances, the wound and the bleeding are minor, and normal blood clotting functions in addition to the application of simple first aid are all that is required. First aid may include applying pressure to the wound with a sponge or similar device to facilitate clotting functions. Unfortunately, however, in other circumstances substantial bleeding can occur. Bleeding can also be a problem when the trauma is the result of a surgical procedure. Apart from suturing or stapling an incision or internally bleeding area, bleeding encountered during surgery is often controlled using sponges or other materials used to exert pressure against the bleed site and/or absorb the blood. However, when the bleeding becomes excessive, these measures may not be sufficient to stop the blood flow.

### SUMMARY

Materials comprising fibers with a macromolecular material and a hemostatic additive material may be useful to promote blood clotting, such as promoting blood clotting in a person or animal that is bleeding. In some embodiments, a material having fibers comprising a macromolecular material, such as a crosslinked macromolecular material, may be useful to promote blood clotting. In some embodiments, the fibers can include a hemostatic additive material. These materials may also be useful for treating chronic wounds or longer term care of wounds such as burns and sores.

In some embodiments, a device for promoting the clotting of blood may comprising a fabric substrate comprising a fibrous material and a sterilized packaging. In some embodiments, the fibrous material may comprise: a host which may comprise a polymer and a hemostatic additive which may comprise a hemostatic agent such as a clay. An example of a suitable clay is kaolin. With respect to structure of the fibrous material, the fibrous material may comprise a plurality of individual fibers, each fiber having an interior and a surface. The hemostatic additive in the fibers may be dispersed throughout the interior of the fibers and affixed to the surface of the fibers. In some embodiments, the surface may have a greater hemostatic additive loading level than the interior. The substrate may also be contained within the sterilized packaging.

Some embodiments may relate to a blood clotting device comprising a sterile gauze substrate comprising a blend of fibers. For example, the substrate may comprise a blend of a first fiber comprising a cellulosic material and a second fiber comprising a synthetic polymer. Furthermore, in some embodiments, at least one of the first fiber and the second fiber may comprise an inorganic additive having hemostatic properties.

Some embodiments relate to a hemostatic device comprising a fibrous material comprising a macromolecular host material and a clay additive material. The fibrous material and/or the additive material may be stable in the presence of blood. In some embodiments, the fibrous material and/or the additive material will not dissolve, disintegrate, or otherwise rapidly degrade when in contact with blood. In some embodiments, either or both of the fibrous material and additive material may disintegrate, dissolve, be released, or leach out in the presence of liquids such as blood.

Some embodiments relate to a hemostatic product comprising a gauze in a sterilized packaging. The hemostatic product may be made by a process comprising: forming a gauze from a plurality of fibers comprising a hemostatic agent such as kaolin and a polymer. The fibers may be formed by a process comprising: blending a mixture comprising the hemostatic agent and the polymer in a molten form and extruding the mixture to produce the fibers upon cooling. Products which are structurally similar to products which may be made by such a process are contemplated regardless of how the products are actually made.

These materials may be used in a variety of methods. For example, any device described herein may be applied to a bleeding wound, or blood issuing from a bleeding wound. In some embodiments, the blood may directly contacted a material, such as a fibrous material, a gauze, or a similar substrate, which comprises fibers comprising any hemostatic additive material.

Some embodiments provide a method of promoting blood clotting comprising: opening a sterilized packaging containing a hemostatic device; and placing the hemostatic device in direct contact with a bleeding area of a human being or animal. The hemostatic device may comprise a fibrous material comprising a macromolecular host material and a clay additive material, and the fibrous material may be stable in the presence of blood.

Some embodiments relate to a method of preparing a hemostatic product comprising: forming a gauze from a plurality of fibers comprising a hemostatic agent such as kaolin and a polymer; wherein the fibers are formed by a process comprising: blending a mixture comprising the hemostatic agent and the polymer in a molten form; extruding the mixture to provide the fibers upon cooling; and providing the hemostatic product comprising the gauze in a sterilized packaging.

### BRIEF DESCRIPTION OF THE DRAWINGS

FIG. 1A is a schematic depiction of a cross section of an embodiment of a fiber having an uniform distribution of a hemostatic device in the fiber.

FIG. 1B is a schematic depiction of a cross section of an embodiment of a fiber having an uneven distribution of a hemostatic device in the fiber.

FIG. 2 is a schematic depiction of a cross section of an embodiment of a fiber, wherein the surface of the fiber has a greater loading level than the interior of the fiber.

FIG. 3 is a schematic of an embodiment of a process which may be used to prepare some embodiments of the fibers described herein.

FIG. 4 depicts a schematic embodiment of a process for exposing hemostatic agent on the surface of a fiber.

### DETAILED DESCRIPTION

The embodiments described herein generally relate to devices and methods for promoting the clotting of blood in human beings or animals. In some embodiments, the devices and methods disclosed herein can be used to promote wound healing, regardless of whether the devices and methods can be used for hemostasis. In some embodiments, the devices may comprise a fibrous material or materials comprising one or more fibers combined to form a substrate such as a textile substrate (e.g., a gauze or a cloth). Some of the fibers in these materials or devices may comprise a macromolecular material and a hemostatic additive material.

A macromolecular material may comprise any macromolecule as broadly understood in the art, and may include any molecule of a large molecular weight, such as molecules having repeating identical or similar units, such as a polymer, a protein or another polypeptide, a carbohydrate based macromolecule, etc.

In some embodiments, the macromolecular material may comprise a polysaccharide such as cellulose, starch, or the like, or a derivative thereof. In some embodiments, the macromolecular material may comprise a cellulosic material as broadly understood by those skilled in the art, including cellulose or modified cellulose, such as cellulose which has been chemically modified by hydrolysis, reaction of the hydroxyl groups, or a combination thereof. Examples include, but are not limited to, regenerated cellulose such as rayon, including lyocell; cellulose esters such as nitrocellulose, cellulose acetates, cellulose priopionates, cellulose butyrate; cellulose ethers such as methylcellulose, ethylcellulose, hydroxyethylcellulose, hydroxypropylcellulose, carboxymethylcellulose, aminoethylcellulose, benzylcellulose, etc.

A polymer may include any polymer as broadly understood by those skilled in the art, and may include a polymer based upon a single repeat unit or a copolymer comprising more than one repeat unit distributed throughout the polymer in any arrangement, such as statistically distributed, or in blocks such as those formed by combining two or more oligomers or polymers of different types. In addition to cellulosic materials, examples of polymers may include, but are not limited to, polyolefins such as polyethylene, polypropylene, polybutylene, poly(4-methyl-1-pentene), and poly(1-hexane), etc.; acrylics such as polyacrylic acid, polymethylacrylate, polymethylmethacrylate, polyacrylonitrile, etc.; other substituted ethylenic polymers such as polyvinylchloride, polyvinyl alcohol, polystyrene, etc.; polyamides such as aliphatic polyamides (e.g. PA 6, PA 66, nylon-6, nylon-66, etc.), polyphthalamides (such as PA 6T), aromatic polyamides (such as a paraphenylenediamine-terephthalic acid polyamides including Kevlar and Nomex), etc.; polyurethanes; polyesters such as polyglycolic acid, polylactic acid, polycaprolactone, polyethylene adipate, polyhydroxyalkanoate, comprising monomer units derived from terephthalic acid such as poly(alkylene terephthalates); poly(alkylene naphthalates), polyethylene terephthalate, polybutylene terephthalate, polytrimethylene terephthalate, polyethylene naphthalate, vectran, polyesters comprising monomer units derived from cyclohexanedimethanol; etc.; polybenzimazole, poly[2,2′-(m-phenylene)-5,5′-bibenzimidazole], etc.; polyalkylene oxides such as polyethylene oxide, polypropylene oxide, polybutylene oxide, etc.; poly(arylene sulfides); etc.

Other macromolecular materials such as protein based fibers, including fibers based upon wool, silk, or modified version of these materials may be used.

Combinations and/or copolymers of one or more polymers or macromolecular materials, such as any polymer or macromolecular material described herein, may be used.

The macromolecular materials may be crosslinked. Under some circumstances, cross-linking the polymer may be useful to improve the strength and/or stability of the macromolecular material, which may also improve the strength and/or stability of the fiber. In some embodiments, the macromolecular material may be crosslinked to provide a molecular sieve which may be useful as a hemostatic device. Thus, in some embodiments, the fiber may comprise a crosslinked polymer which is incorporated into a fibrous material for a hemostatic device without the need for a separate hemostatic additive to provide hemostatic properties. For example, crosslinked polystyrene or a crosslinked hydrophilic polymer such as a hydrophilic acrylic, a hydrophilic alkylene oxide, a hydrophilic polyurethane, a hydrophilic polyamide, a hydrophilic polyester, polyvinyl alcohol, or the like, may be useful a hemostatic material without a separate hemostatic additive. Crosslinkers for various types of polymers and a variety of crosslinked polymers are commercially available and known in the art.

In some embodiments, the blood clotting device or hemostatic device may comprise a fibrous material such as a gauze or a cloth, which may comprise a blend of two or more fibers, such as a blend of a first fiber comprising a cellulose derivative and a second fiber comprising polymer such as a synthetic polymer. In these embodiments, at least one of these fibers may comprise a hemostatic additive having hemostatic properties. In some embodiments, a fiber may comprise a cellulose derivative, such as rayon, and a hemostatic additive having hemostatic properties. In some embodiments, a fiber may comprise a polyurethane, a polyamide, a polythiazole, or a polyester and a hemostatic additive having hemostatic properties. In some embodiments, the fibrous material may comprise a blend of polyester fibers and rayon fibers. The combination of polyester and rayon may help to provide qualities such as softness, conformability, strength, and absorbancy, which may be useful for a wound dressing. In some embodiments, the fibrous material comprises a blend of polyester fibers and rayon fibers, and the polyester fibers comprise a hemostatic additive such as kaolin or another clay. In some embodiments, the fibrous material comprises a blend of polyester fibers and rayon fibers, and the rayon fibers comprise a hemostatic additive such as kaolin or another clay.

A hemostatic additive may be present in any fiber described herein. A hemostatic hemostatic additive material may include any hemostatic material, including inorganic hemostatic materials such as silicates, including bioactive glass, silica gel (SiO₂); alumina gel (Al₂O₃); silica-alumina, SiO₂—Al₂O₃; and silica-calcia SiO₂—CaO, and the like; aluminosilicates, such as zeolites including (Na) zeolite 4A, ((Na)₁₂[(AlO₂)₁₂(SiO₂)₁₂].27H₂O), available under the trade name PURMOL 4A from Zeochem of Louisville, K.Y., (Ca) zeolite 5A ((Ca)₆[(AlO₂)₁₂(SiO₂)₁₂].27H₂O), available under the trade name LINDE TYPE A from Union Carbide (Na) zeolite 4A ((Na)₁₂[(AlO₂)₁₂(SiO₂)₁₂].27H₂O Y ((Na)₅₆(Al₅₆Si₁₃₆O₃84).250H₂O)), (Ca) zeolite Y ((Ca, Na)₅₆(Al₅₆Si₁₃₆O₃₈₄).250H₂O)), (K) OMS-2 ((K)Mn₈O₁₆.nH₂O) (Ca) OMS-2 ((Ca, K)Mn₈O₁₆.nH2O), MgₓAl_{y}(OH)_{z}Clᵤ.nH₂O, chabazite (K₁₁(Al₁₁Si₂₅O₇2).40H₂O), (Ca) OL-1 ((Ca,K,Na)Mn₁₄O₂₇.21H₂O), ZSM-5 (Na₇(Al₇Si₈₉O₁₉₂).nH₂O), (Ca) ZSM-5 (Ca,Na)₇(Al₇Si₈9O₁₉₂).nH₂O) zeolite RHO, (Na,CS)₁₂[Al₁₂Si₃₆O₉₆].44H₂O, Ca-mordenite, (Na—Ca)₅[Al₅Si₄₃O₉₆].nH₂O; or other inorganic molecular sieves, or clays, including clays such as kaolin, bentonite, montmorillonite, saponite, polygorskite or attapulgite, sepiolite, etc.; oxides such as sodium aluminum oxide (NaAlO₂), available from Alfa Aesar Company of Ward Hill, Mass., magnesium oxide (MgO), acid silica (SiO₂.nH₂O), titanium dioxide (TiO₂), available as TITANIC OXIDE, and barium oxide (BaO), all available from Fisher Scientific Company, activated carbon, available from Strem Chemicals of Newburyport Mass., europium oxide (Eu₂O₃) and cerium oxide (CeO₂), available from American Potash & Chemical Corp. of West Chicago, Ill., copper oxide (CuO), available from Cerac Inc. of Milwaukee, Wis., cobalt oxide (CO₂O₃), available from J. T. Baker, bismuth oxide (Bi₂O₃), available from Baker and Adamson Chemical Co., aluminum oxide (Al₂O₃), available as aluminum oxide neutral type T from EM Reagents, nickel oxide (NiO), available from Matheson, Coleman and Bell of East Rutherford, N.J., zinc oxide (ZnO), stannic oxide (SnO₂), and iron oxide, (Fe₂O₃) all available from Baker Analyticals, manganese oxide (MnO), available as manganese IV oxide, 99% and zirconium (IV) oxide (ZrO₂), all available from Aldrich, vanadium pentoxide (V₂O₅), available from Mallinkrodt, scandium oxide (Sc₂O₃), available as scandium oxide 98% from A. D. Mackay of New York, yttrium oxide (Y₂O₃), available from Alfa Aesar Company of Ward Hill, Mass., CHROMOSORB P-AW-DMCS, CHROMOSORB 101, and CHROMOSORB 102, all available from Alltech Associates, Deerfield Ill.; calcium salts such as calcium oxide (CaO), calcium chloride (CaCl₂), dibasic calcium phosphate (CaHPO₄), etc.; activated carbon, etc. Other hemostatic agents may include biologically based hemostatic agents such as chitosan. The hemostatic additive may also be an organic molecular sieve, such as crosslinked polystyrene or a crosslinked hydrophilic polymer such as a hydrophilic acrylic, a hydrophilic alkylene oxide, a hydrophilic polyurethane, a hydrophilic polyamide, a hydrophilic polyester, polyvinyl alcohol, or the like. These cross-linked macromolecules may also be useful as a hemostatic material without requiring a separate hemostatic additive.

In some embodiments the hemostatic agent may be a clay, such as a kaolin clay, which includes the mineral “kaolinite.” The kaolin may be Edgar's plastic kaolin (hereinafter “EPK”), which is a water-washed kaolin clay that is mined and processed in and near Edgar, Fla. Edgar's plastic kaolin may have desirable plasticity characteristics, may be castable, and when mixed with water may produce a thixotropic slurry.

The amount of the hemostatic additive material may vary. In some embodiments, the weight of the hemostatic additive material as compared to the weight of the overall fiber may be at least about any of the following: 1%, 2%, 5%, or 7%; and/or less than or equal to about any of the following: 15%, 20%, 30%, 50%, or 75%. For example, the weight of the hemostatic additive may be about 10% of the weight of the fiber. In some embodiments, the particle size, such as the particle size of a clay such as a kaolin, may be less than or equal to about any of the following: 50 microns, 20 microns, or 2 microns. In some embodiments at least about 99% of the particles are less than about 50 microns. In some embodiments, at least about 80% of the particles are less than 10 microns. In some embodiments, at least about 20%, at least about 45%, or at least about 80% of the particles are under 2 microns.

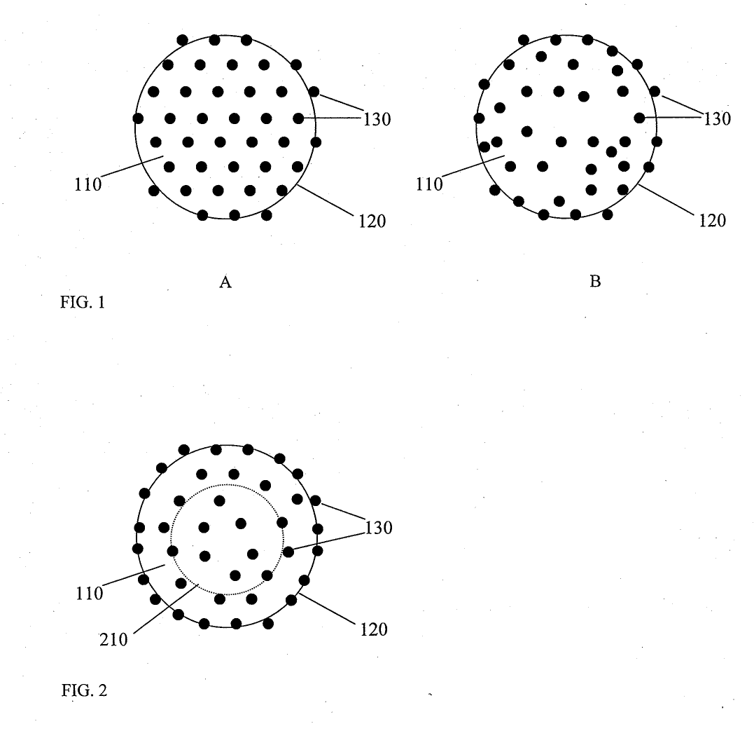

FIG. 1 depicts a cross section of some embodiments of a fiber. In FIG. 1, a fiber may have an interior 110 and a surface 120, and the hemostatic material 130, such as kaolin, may be dispersed throughout at least a portion of (or generally all of) the interior 110 of the fiber. Thus, the fiber may be a composite material comprising a solid macromolecular host with the hemostatic additive material mixed throughout the solid fiber. The dispersion of the hemostatic material in the macromolecular host may be generally uniform, as shown in FIG. 1A, or may be generally uneven, as depicted in FIG. 1B. The hemostatic material 130 may also be affixed to the surface 120 of the fiber. In some embodiments, the hemostatic material 130 may be affixed to the surface 120 of the fiber and/or not generally contained within the interior of the fiber. In some embodiments, the hemostatic material may be affixed so that does not become separated from the fiber surface in the presence of blood. In some embodiments, the hemostatic agent may remain attached or adhered to the fiber surface for at least about 5 minutes, at least about 10 minutes, at least about 60 minutes, or at least about 24 hours.

In some embodiments, the surface of the fiber may have a greater loading level than the interior of the fiber. FIG. 2 depicts an example of an embodiment of a fiber wherein the surface of the fiber has a greater loading level than the interior of the fiber. The loading level of the surface may be compared to the loading level in the interior by a variety of methods. While not limiting any embodiment, one convenient method may be to draw a concentric circle 210 inside the fiber cross section and compare the relative loading of the hemostatic additive particles on the inside circle 210 as compared to the circle formed by the surface 120. If the particles are generally uniform in size, the particles themselves can be counted and divided by the circumference of the appropriate circle. If the particles are not of generally uniform size, the areas of the particles on each circle could be totaled and divided by the circumference of the appropriate circle.

The fiber may be of any thickness which is appropriate for a fibrous material comprising a hemostatic material. In some embodiments, the fiber diameter may be at least about any of the following: 0.1 μm, 1 μm, 2 μm, 3 μm, or 5 μm; and/or less than about any of the following: 45 μm, 50 μm, 60 μm, or 80 μm. In some embodiments, the fiber diameter may be about 0.1 μm to about 100 μm, about 0.1 μm to about 50 μm, about 1 μm to about 45 μm, about 3 μm to about 71 μm, or about 40 μm to about 100 μm. In some embodiments, the fiber is substantially stable in the presence of blood. The term “stable” is intended to have the meaning generally understood in the art, and includes a fiber which does not disintegrate or dissolve in the presence of blood during the time that it generally takes the blood to clot in a particular type of application. In some embodiments, the fiber remains generally intact, and does not disintegrate or dissolve at all in the presence of blood, or until after at least about 5 minutes, at least about 10 minutes, at least about 60 minutes, or at least about 24 hours, of contact with blood. In some embodiments, the fiber is sufficiently stable that the hemostatic material is not lost from the fiber by disintegration or dissolution of a macromolecular material in the fiber. For example, in some embodiments, the fiber is configured so that if the fiber comes in contact with blood, substantially all of the hemostatic material, such as kaolin or another clay, is retained in or on the fiber for at least about 5 minutes, at least about 10 minutes, at least about 60 minutes, or at least about 24 hours while the fiber is in contact with blood. In some embodiments, the hemostatic material, such as a clay (including kaolin), is affixed to the surface so that substantially all of the particles of the hemostatic material are retained and no clinically significant amount of particles become detached from the fiber. For example, in some embodiments, the fiber is configured so that if the fiber comes in contact with blood, the particles of hemostatic material do not become detached for at least about 5 minutes, at least about 10 minutes, at least about 60 minutes, or at least about 24 hours while the fiber is in contact with blood.

In some embodiments, the hemostatic additive, such as kaolin or another clay, may be uncoated. However, in some embodiments, the hemostatic additive may be coated with an organic material such as a fatty acid, such as stearic acid or a stearate salt, including calcium stearate or ammonium stearate. For some extrusion processes, a coating may help the hemostatic additive to remain in the interior of the fiber. A hemostatic additive without coating may be useful if higher loading is desired at the surface of the fiber, or for other reasons.

A fiber may contain other additives such as a therapeutically active agent such as an analgesic, including but not limited to, an opiate such as codeine, morphine, oxycodone, etc.; acetaminophen; anti-inflammatory agents, including nonsteroidal anti-inflammatory drugs, aspirin, etc.; an antibiotic or another antimicrobial drug or compound; an antihistamine (e.g., cimetidine, chloropheniramine maleate, diphenhydramine hydrochloride, and promethazine hydrochloride); antifungal agents; anti-microbial compounds such as those containing silver ions; compounds containing copper ions; ascorbic acid; tranexamic acid; rutin; thrombin; botanical agents; etc.; and combinations thereof. Other additives may include magnesium sulfate, sodium metaphosphate, calcium chloride, dextrin, and combinations thereof.

The fiber may be formed by a variety of methods. Some methods can include the general method shown in schematic form in FIG. 3. First, the macromolecular material, the hemostatic additive, and any other constituents, may be combined in liquid or slurry form 310. Next, the liquid or slurry may be extruded 320. The extruded material can then be allowed to solidify or harden to form the solid fiber 330.

There are several ways that the macromolecular material and the hemostatic additive may be combined in liquid or slurry form. For example, the hemostatic additive may be combined with a macromolecule or polymer in a molten form. When the molten mixture is extruded, fibers may form upon cooling. For example, the cooling may cause the molten macromolecule or polymer to cool to a solid, thus forming a solid fiber incorporating the hemostatic additive material. The hemostatic additive may also be combined with the macromolecule or polymer and water or an organic liquid which dissolves or disperses the macromolecule or polymer, so that the mixture can be thoroughly blended into a liquid or a slurry. The liquid or slurry may then be extruded, and form fibers upon evaporation of the liquid. Suitable organic liquids may include, but are not limited to, ethanol, methanol, isopropanol, ethyl ether, dichloromethane, butane, pentane, hexane, heptane, acetone, ethyl acetate, and the like.

In some embodiments, the polymer may be formed in the presence of the hemostatic additive material such as kaolin and extruded before the material cures. For example, a liquid monomer such as a low molecular weight olefin, acrylic acid, methyl methacrylate, acrylonitrile, etc., may be combined with the hemostatic additive material, and then the polymerization reaction may be initiated. When the polymerization reaction has been initiated, the material may be extruded while still in the liquid state, and the fibers may form as the polymerization reaction progresses or the material cures. A similar process could be carried out with polymer made from two or more components such as polyesters, polyurethanes, etc.

The extrusion may be carried out with any type of die or other extrusion equipment ordinarily used to produce fibers. In some embodiments, the fibers are produced by extruding the liquid or slum through spinerette dies. For example, the extrusion may be carried out using DuPont Fiber spinning equipment. In some embodiments, the fibers may be drawn as they are extruded. Drawing, including pulling on the polymer fiber as it exits the extruder die, may help to improve the strength of a polymer fiber.

The die may have any diameter, in some embodiments, the die diameter may be from about 0.01 mm to about 5 mm in diameter, about 0.05 to about 1 mm in diameter, about 0.5 mm to about 0.6 mm in diameter, about 0.6 mm to about 0.7 mm in diameter, about 0.7 mm to about 0.8 mm in diameter, about 0.8 mm to about 0.9 mm in diameter, about 0.9 mm to about 1 mm in diameter, about 1 mm to about 1.5 mm in diameter, about 1.5 mm to about 2 mm in diameter, about 2 mm to about 2.5 mm in diameter, about 2.5 mm to about 3 mm in diameter, or about 3 mm to about 4 mm in diameter.

The extrusion rate and temperature may vary. For melt extrusion processes, in some embodiments the temperature may range from about 50° C., about 20° C., or about 10° C. below the softening point or melting point of the polymer to about 10° C., about 20° C., or about 50° C. above the softening point or melting point of the polymer. As mentioned above, coating may help particles to remain in the interior of the liquid or slurry as opposed to providing greater levels at the surface or interface during the extrusion processes. Coating may be avoided in certain circumstances by using a macromolecule that interacts well with the hemostatic additive. For example, since kaolin and other clays tend to be minerals with charges, they may mix better with more ionic polymers or macromolecules, such as polyacrylic acid, carboxymethylcellulose, or the like, or with more polar polymers or macromolecules such as polyamides, polyurethanes, polyvinyl alcohol, polysaccharides, hydroxypropylmethylcelullose, some polyesters, etc. For less polar hemostatic agents, such as crosslinked polystyrene, a less polar macromolecular material, such as a polyolefin, may be used.

For embodiments where the fiber has a greater density or loading of the hemostatic additive at the surface of the fiber than in the interior of the fiber, the hemostatic additive particles may make extrusion more difficult. To incorporate a fine powder hemostatic additive into rayon fibers or other polymer fibers, a spinneret may be modified from a standard design to improve the ability to extrude the fibers. For example, a powder additive may clog the holes in a standard spinneret, so holes with larger diameters than standard spinnerets, such as about 0.6 to about 1 mm in diameter, about 0.5 mm to about 0.6 mm in diameter, about 0.6 mm to about 0.7 mm in diameter, about 0.7 mm to about 0.8 mm in diameter, about 0.8 mm to about 0.9 mm in diameter, about 0.9 mm to about 1 mm in diameter, about 1 mm to about 1.5 mm in diameter, about 1.5 mm to about 2 mm in diameter, about 2 mm to about 2.5 mm in diameter, about 2.5 mm to about 3 mm in diameter, or about 3 mm to about 4 mm in diameter, may be used to facilitate extrusion under these circumstances. The profile or longitudinal cross section of each hole may be optimized to enable flow of the cellulose or other polymer and a hemostatic powder. Other variables that may be adjusted include the surface finish (roughness) or coating inside the holes, the number of holes per spinneret, and the material selection (e.g. type of metal alloy) for the spinneret.

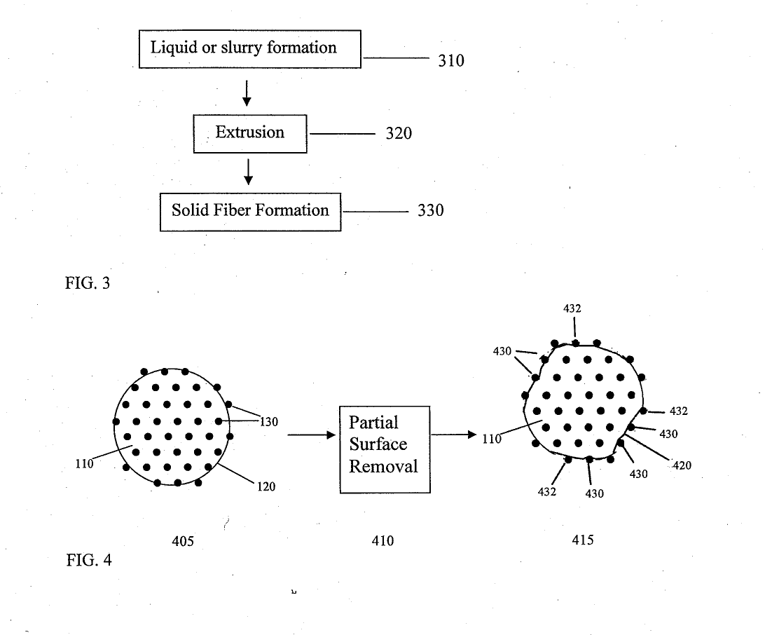

If desired, the amount of hemostatic agent exposed on the surface of the fiber may be increased by a surface treatment, such as removal of at least part of the surface of the fiber to expose more of the hemostatic agent. An example of this is depicted in FIG. 4. In this example, a fiber 405 with an untreated surface 120 is subject to partial surface removal 410 to provide a treated fiber 415 with an enhanced surface 420 in which substantially more surface area, as a proportion or percentage of the total surface area, is comprised of hemostatic agents than before the surface treatment. As result of the partial surface removal 410, the treated fiber 415 has exposed hemostatic agent particles which are either newly exposed 430 or have greater exposure 432 on the enhanced surface 420, as compared to the untreated fiber 405. The partial surface removal may be accomplished by many different methods, such as chemical etching, including chemical etching by a solvent such as ethanol, methanol, isopropanol, ethyl ether, dichloromethane, butane, pentane, hexane, heptane, acetone, ethyl acetate, and the like, oxidizing agents such as oxygen, ozone, hypochlorite, hydrogen peroxide, peroxy acids, permanganate, perchlorate, etc.; applying acids such as hydrochloric acid, hydrofluoric acid, sulfuric acid, acetic acid, trifluoroacetic acid, trifluoromethanesulfonate, nitric acid, phosphoric acid, etc.; and applying bases such as aqueous sodium hydroxide, or other agents. Mechanical methods, such as microcreping, may also be used. Chemical methods and mechanical methods may also be combined. Under some circumstances, the treating may also be carried out on a fibrous material such as a cloth or a gauze instead of the fibers. In some embodiments, performing the surface modification treatment on a textile, such as a gauze, cloth, or fabric, after the fibers have been woven or otherwise formed into a textile, may facilitate the process of textile formation and/or the strength of the textile. In some embodiments, the surface modification treatment can be performed on the fibers before forming the textile.

Once the fibers are formed, they may be woven into fibrous material or a substrate such as a gauze or a cloth. Alternatively, the fibrous material or substrate may be formed by a nonweaving method such as spunlace, needlepunch, or the like.

The fibrous material may be in the form of a gauze or cloth. In some embodiments, the gauze or cloth may have a thickness of at least about any of the following: 0.01 mm, 0.5 mm, or 1 mm, to 2 mm, 3 mm, or 5 mm. In some embodiments, the gauze or cloth may be formed into a roll with a width of at least about 1 inch or at least about 2 inches to about 5 inches, about 10 inches, about 2 feet, about 3 feet, about 6 feet, or about 10 feet. In some embodiments, a nonwoven fabric, such as a cloth or gauze may be formed into a roll having a width of about 1 to about 10 feet or about 2 to about 6 feet. In some embodiments, the gauze or cloth may be at least about 3 inches wide. The length of the roll may be at least about any of the following: 0.5 yards, 1 yard, or 3 yards; and/or less than or equal to about any of the following: 5 yards, 10 yards, or 20 yards. In some embodiments, the roll is about 3 inches by about 4 yards. In some embodiments, a gauze or cloth of the dimensions described above may be folded into pleated, or “Z” form. In some embodiments, the gauze or cloth may be cut or otherwise formed into smaller pieces having a length of at least about 1 inch or at least about 2 inches to about 5 inches or about 10 inches, and a width of least about 1 inch or at least about 2 inches to about 5 inches or about 10 inches, and having a generally square or generally rectangular shape. In some embodiments, the cloth or gauze is about 2 inches by about 2 inches.

In some embodiments, the fibrous material is sufficiently porous to allow blood to readily penetrate the outer layer of fibers, thus allowing more complete contact between blood and the surface of the hemostatic fiber. In some embodiments, the fibrous material is a gauze of cloth having pores which may be at least about 0.01 mm, about 0.1 mm, about 0.2 mm, or about 0.3 mm, to about 0.5, about 1 mm, about 1.5 mm, or about 2 mm. In some embodiments, the pores may be about 0.01 to about 0.1 mm, about 0.1 mm to about 0.3 mm, about 0.3 mm to about 0.5 mm, about 0.5 mm to about 0.8 mm, about 0.8 mm to about 1 mm, about 1 to about 1.5 mm, or about 1.5 to about 2 mm.

In some embodiments, it may be useful to further coat the fibers or the fibrous material with additional hemostatic agent that is the same as or different from to that used in the fibrous material. In some embodiments, a binder may be useful to help the hemostatic agent to adhere to the fibrous material, or to bind the hemostatic agent to the fibrous material. In some embodiments, a binder is a substance which is similar to the macromolecule or the hemostatic agent. It may be helpful for the binder to be a liquid. For example, oligomers of the macromolecule or polymer in some of the fibers may be useful as binders. For inorganic hemostatic agents and polar fibrous materials, the binder may have some polar groups or hydrogen bonding groups such as hydroxyl, amino, ether, carbonyl, or the like. Examples of useful binders may include, but are not limited to, polyols having a formula HOCH₂(CHOH)ₙCH₂OH, wherein n is 0, 1, 2, 3, 4, 5, 6, 7, 8, 9, or 10, including glycerin and other sugar alcohols, C₃₋₈ alcohols such as butanol, pentanol, etc., polymeric polyols such as polyvinyl alcohol, polysaccharides and derivatives thereof such as guar gum, gelatinized starches, cellulose, alginic acids and salts thereof such as calcium aginate, chitosan, carboxymethyl cellulose, hydroxypropylmethylcellulose, etc.

In some embodiments, the hemostatic or blood clotting device may comprise a release agent disposed on a fibrous material or a substrate. The release agent may be any material which helps the hemostatic agent to be more easily removed after use. In some embodiments, the release agent may be a material with low adhesion to skin or other body tissue. Examples of useful release agents may include, but are not limited to, polyols having a formula HOCH₂(CHOH)ₙCH₂OH, wherein n is 0, 1, 2, 3, 4, 5, 6, 7, 8, 9, or 10, including glycerin and other sugar alcohols, C₃₋₈ alcohols such as butanol, pentanol, etc., polymeric polyols such as polyvinyl alcohol, polysaccharides and derivatives thereof such as guar gum, gelatinized starches, cellulose, alginic acids and salts thereof such as calcium aginate, chitosan, carboxymethyl cellulose, hydroxypropylmethylcellulose, etc; silicon based materials such as silicone; fluorocarbons such as polytetrafluorethylene; and triglycerides such as vegetable oils, and derivatives thereof.

The fibrous material, which may comprise a gauze or a fabric type substrate may be directly used as a hemostatic device. The fibrous material may be coupled to other features or components typically associated with stopping bleeding. For example, the fibrous material may comprise a pressure component, which may be used to apply direct pressure by including a tie or strap which wraps around a body part, a stiff backing to apply pressure to an area, an inflatable feature such as a balloon which may be useful to apply pressure to the interior of a wound. The fibrous material may also comprise an attachment component, which helps the hemostatic material to remain in place at the bleeding area. For example, an adhesive strip, a tie or strap, or a wrap or covering comprising a flexible material such as an elastic may be included.

In some embodiments, the hemostatic device may be sterilized and/or packaged in a sterile or sterilized packaging. Vacuum packing the devices in the packaging may help to reduce the size of the packaging and thus facilitate shipping and storage of the products.

Although the claims have been described in the context of certain preferred embodiments and examples, it will be understood by those skilled in the art that the scope of the claims extend beyond the specifically disclosed embodiments to other alternative embodiments and/or uses and obvious modifications and equivalents thereof.

What is claimed is:

1. A method of promoting blood clotting comprising:
opening a sterilized packaging containing a hemostatic device; and
placing the hemostatic device in direct contact with a bleeding area of a human being or animal;
wherein the hemostatic device comprises a fibrous material comprising a macromolecular host material and a clay additive material; and
wherein the fibrous material is stable in the presence of blood.

2. The method of claim 1, wherein the clay additive material comprises kaolin.

3. The method of claim 1, wherein the clay additive is uncoated.

4. The method of claim 1, wherein the macromolecular host material comprises rayon or a polyester.

5. A device for promoting the clotting of blood comprising:
a fabric substrate comprising a fibrous material and a sterilized packaging
wherein the fibrous material comprises: a host comprising a polymer, and a hemostatic additive comprising kaolin;
wherein the fibrous material comprises a plurality of individual fibers, each fiber having an interior and a surface;
wherein the kaolin is dispersed in the host throughout the interior and affixed to the host at the surface;
wherein the surface has a greater loading level of kaolin than the interior; and
wherein the substrate is contained within the sterilized packaging.

6. The device of claim 5, wherein the polymer comprises rayon.

7. The device of claim 5, the plurality of fibers is substantially stable in the presence of blood.

8. The device of claim 5, wherein the fabric substrate has a length of about 1 inch to about 10 inches and a width of about 2 inches to about 5 inches.

9. The device of claim 5, wherein the fabric substrate has a length of about 0.5 yards to about 20 yards and a width of about 1 inch to about 10 inches.

10. A blood clotting device comprising:
a sterile gauze substrate comprising a blend of a first fiber comprising a cellulosic material and a second fiber comprising a synthetic polymer; and
wherein at least one of the first fiber and the second fiber comprises an inorganic hemostatic additive having hemostatic properties.

11. The device of claim 10, wherein the cellulosic material comprises rayon and the synthetic polymer comprises a polyester.

12. The device of claim 10, wherein devices is configured so that if the first fiber or the second fiber comes in contact with blood, substantially all of the inorganic hemostatic additive is retained in or on the first fiber or the second fiber for at least about 10 minutes of contact with blood.

13. The device of claim 10, wherein the second fiber comprises the inorganic hemostatic additive.

14. The device of claim 10, wherein the first fiber comprises the inorganic hemostatic additive.

15. The device of claim 10, wherein the inorganic hemostatic additive is about 1% to about 20% of the second fiber by weight.

16. The device of claim 10, wherein the first fiber has a diameter of about 0.1 μm to about 100 μm.

17. The device of claim 10, wherein at least one of the first fiber and the second fiber comprises a therapeutically active agent.

18. The device of claim 10, further comprising a release agent disposed on the gauze substrate.

19. The device of claim 10, wherein the device is vacuum packed in a sterile or sterilized packaging.

20. The device of claim 10, wherein the first fiber is rayon and the inorganic hemostatic additive is kaolin.
